(19) United States
(12) Patent Application Publication
(10) Pub. No.: US 2024/0344824 A1
(43) Pub. Date: Oct. 17, 2024

(54) SINGLE PATTERN SHIFT PROJECTION OPTICAL SYSTEM FOR 3D SCANNER

(71) Applicant: Medit Corp., Seoul (KR)

(72) Inventor: Eun Gil CHO, Seoul (KR)

(73) Assignee: Medit Corp., Seoul (KR)

(21) Appl. No.: 18/754,134

(22) Filed: Jun. 25, 2024

Related U.S. Application Data
(63) Continuation of application No. PCT/KR2022/019881, filed on Dec. 8, 2022.

(30) Foreign Application Priority Data
Dec. 28, 2021 (KR) 10-2021-0189447

(51) Int. Cl. G01B 11/25; A61B 1/00; A61B 1/24; G02B 5/04

(52) U.S. Cl. CPC G01B 11/2518; G02B 5/04; A61B 1/00172; A61B 1/24

(57) ABSTRACT

The present disclosure relates to a single pattern shift projection optical system for a 3D scanner, which shifts and projects a pattern image for 3D scanning.

The single pattern shift projection optical system for a 3D scanner of the present disclosure comprises: a plurality of light sources each emitting light in different constant wavelength bands; a pattern member that transmits only a portion of the light generated from each of the plurality of light sources as patterned light; and a refracting member disposed on a path of light passing through the pattern member to refract and transmit the patterned light.

### CROSS-REFERENCE TO RELATED APPLICATION

This application is a Continuation of PCT international application No. PCT/KR2022/019881, filed on Dec. 8, 2022, which claims the priority benefit of Korea application no. 10-2021-0189447, filed on Dec. 28, 2021. The entirety of each of the above mentioned patent applications is hereby incorporated by reference herein and made a part of this specification.

### TECHNICAL FIELD

The present disclosure relates to a single pattern shift projection optical system for a three-dimensional (3D) scanner that shifts and projects a pattern image for 3D scanning.

### BACKGROUND

There are various 3D scanning methods for obtaining 3D shape data using two-dimensional (2D) images.

A 3D shape of an object may be computed by capturing images of the object from various angles using a single camera, and a 3D shape may also be computed using photographic images captured by a plurality of cameras whose relative positions are known.

In this method, a commonly used method is to find feature points to specify the same portions of an object in a 2D image and match the feature points. The accuracy and quality of 3D shape data is determined by how accurately and how many of these feature points are acquired.

When a 2D image is captured to compute a 3D shape of an object in this manner, image capturing methods of projecting a pattern image onto the object are widely used.

Such methods of projecting a pattern image include a method of projecting a single pattern and a method of projecting a plurality of patterns.

The method using a plurality of patterns generally uses an optical modulator (i.e., a spatial modulator) or a piezoelectric actuator to generate a pattern change. In particular, phase-shifting technology, which is widely used in 3D measurement, requires three or more pattern images of different phases. An optical modulator or a piezoelectric actuator used for this purpose may divide the phase of a pattern into small pieces to generate many pattern images. In particular, optical modulators may easily produce complex patterns, and thus are widely used in other 3D measurement algorithms in addition to phase-shifting technology.

Optical modulators have the problem of having a complex structure, requiring a number of essential elements, and requiring a complex control circuit and driving software. Piezoelectric actuators have the problem that micro-vibrations may occur during operation, thereby causing a deviation in 2D images.

Especially for small real-time 3D scanners, such as dental scanners, there is the problem of significant structural limitations such as increased volume and weight when using an optical modulator or a piezoelectric actuator.

Therefore, when a pattern image projection optical system that may shift and project a pattern image while having a relatively simple structure without using an optical modulator or a piezoelectric actuator is developed, a 3D scanner may be fabricated smaller in size and lighter in weight.

The purpose of the present disclosure is to provide a single pattern shift projection optical system for a 3D scanner that may effectively project a shifted pattern image while having a simple structure for 3D scanning.

### SUMMARY

### Technical Solution

A single pattern shift projection optical system for a 3D scanner according to the present disclosure includes: a plurality of light sources configured to emit light in different constant wavelength bands, respectively; a patterning member configured to transmit a portion of the light generated by each of the plurality of light sources as patterned light; and a refracting member disposed on a path of light passing through the patterning member and configured to refract and transmit the patterned light.

### Advantageous Effects

The single pattern shift projection optical system for a 3D scanner of the present disclosure has the advantage of shifting and projecting a pattern image using a simple structure and a control circuit.

The single pattern shift projection optical system for a 3D scanner of the present disclosure has the advantage of reducing power consumption and easy application to a wireless 3D scanner due to a simple structure and easy fabrication in a small size.

In addition, the single pattern shift projection optical system for a 3D scanner of the present disclosure has the advantage of reducing the weight of a 3D scanner, thereby enabling a 3D scanner that is easy to carry.

In addition, the single pattern shift projection optical system for a 3D scanner of the present disclosure has the advantage of a simple structure, thereby enabling a 3D scanner to be fabricated at low cost and in various shapes without restrictions.

### DESCRIPTION OF DRAWINGS

FIG. 1 is a schematic diagram of a single pattern shift projection optical system for a 3D scanner according to a first embodiment of the present disclosure.

FIG. 2 is a diagram for explaining the operation of a portion of the single pattern shift projection optical system for a 3D scanner shown in FIG. 1.

FIG. 3 is a schematic diagram of a single pattern shift projection optical system for a 3D scanner according to a second embodiment of the present disclosure.

FIG. 4 is a diagram for explaining the operation of a portion of the single pattern shift projection optical system for a 3D scanner shown in FIG. 3.

FIG. 5 is a schematic diagram of a single pattern shift projection optical system for a 3D scanner according to a third embodiment of the present disclosure.

### DETAILED DESCRIPTION

Hereinafter, with reference to the accompanying drawings, a single pattern shift projection optical system for a 3D scanner according to embodiments of the present disclosure will be described in detail.

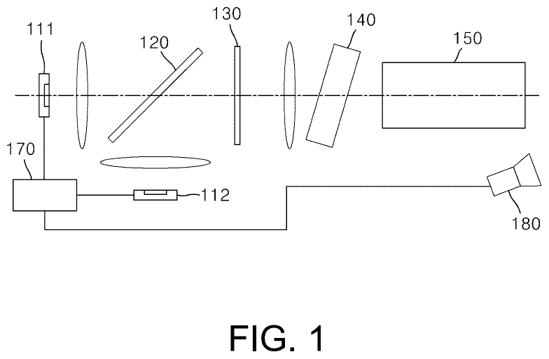

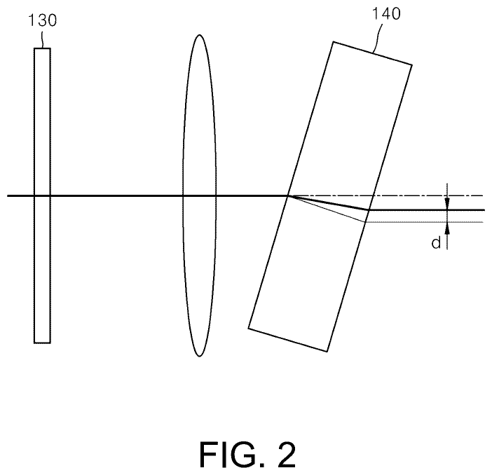

FIG. 1 is a schematic diagram of a single pattern shift projection optical system for a 3D scanner according to a first embodiment of the present disclosure, and FIG. 2 2 is a diagram for explaining the operation of a portion of the single pattern shift projection optical system for a 3D scanner shown in FIG. 1.

Referring to FIGS. 1 and 2, the single pattern shift projection optical system for a 3D scanner of this embodiment includes a plurality of light sources 111 and 112, an incident path member 120, a patterning member 130, and a refracting member 140.

The plurality of light sources 111 and 112 are configured such that each of the light sources 111 and 112 emits a predetermined wavelength band of light and the wavelengths of light emitted from the respective light sources 111 and 112 are different from each other. It is desirable that the wavelength of light generated by each of the light sources 111 and 112 is constant, but each of the light sources 111 and 112 may be configured to emit light in a frequency band within a predetermined range that may be regarded as a substantially constant wavelength depending on the configuration of the light sources 111 and 112.

In this embodiment, as shown in FIG. 1, two different light sources 111 and 112 that emit blue light and red light, respectively, are provided. Since light generated by the respective light sources 111 and 112 is blue light and red light, the light sources 111 and 112 generate different wavelengths of light, respectively. As described above, it is desirable that the wavelength of light generated by each of the light sources 111 and 112 has a predetermined constant value, but in some cases, each of the light sources 111 and 112 may be configured to generate light within a range that may be regarded as substantially the same wavelength. In this embodiment, LED lamps configured to generate blue light and red light, respectively, are used as the light sources 111 and 112. In some cases, a light source may be implemented using a lamp configured to generate white light and a filter configured to transmit only a specific frequency band of light.

The patterning member 130 transmits only a portion of light generated by each of the plurality of light sources 111 and 112 in the form of a pattern. Generally, the patterning member 130 is configured in the form of a pattern mask. In this embodiment, a case in which the patterning member 130 includes a plurality of slits configured to extend in a horizontal direction and arranged parallel to each other at regular intervals will be described as an example. The pattern projected by the patterning member 130 is for computing a 3D shape of an object, and may be modified as needed into structures capable of projecting various types of patterns, such as a sinusoidal shape, a concentric circle shape, a grid pattern shape, in addition to a slit structure in the form of a straight line.

Referring to FIG. 1, in the single pattern shift projection optical system for a 3D scanner of this embodiment, an incident path member 120 is disposed between the light sources 111 and 112 and the patterning member 130. The incident path member 120 is configured to adjust the optical path of light generated by each of the light sources 111 and 112 so that the light is incident on the patterning member 130 in the same direction.

In this embodiment, as shown in FIG. 1, the light source 111 emitting blue light is disposed to irradiate light in a direction perpendicular to the patterning member 130, and the light source 112 emitting red light is disposed in a direction perpendicular to the blue light source 111.

The incident path member 120 is configured in the form of flat glass disposed to be tilted and is disposed between the light sources 111 and 112 and the patterning member 130. The incident path member 120 transmits blue light in the same direction as the incident direction. The incident path member 120 reflects red light in a direction bent by 90 degrees. Accordingly, the incident path member 120 functions to adjust the optical paths of light generated by the light sources 111 and 112 so that blue light and red light generated by the respective light sources 111 and 112 are incident on the patterning member 130 in the same direction. The incident path member 120 described above may be implemented using various types of optical devices depending on the type and arrangement of the light sources 111 and 112.

The refracting member 140 is disposed on the path of patterned light passing through the patterning member 130. The patterning member 130 is configured to refract and transmit light. In this embodiment, as shown in FIGS. 1 and 2, the refracting member 140 configured in the form of transparent flat glass is disposed on the path of light passing through the patterning member 130. In addition, the refracting member 140 is disposed to be tilted with respect to the path of light passing through the patterning member 130. Since the flat glass type refracting member 140 is disposed to be tilted in this manner, the light pattern formed by the patterning member 130 is refracted at a predetermined angle according to the wavelength of the light and the refractive index of the refracting member 140 before passing through the refracting member 140.

In addition, the two light sources 111 and 112 described above are each connected to a controller 170 and turning-on thereof are controlled by the same. The controller 170 alternately turns off the two light sources 111 and 112. That is, in response to the controller 170 alternately turning off the two light sources 111 and 112, red light and blue light are alternately incident on the patterning member 130.

A projector 150 is disposed in front of the refracting member 140. The projector 150 allows the light pattern that has passed through the refracting member 140 to be projected onto the object to be 3D scanned. The projector 150 enlarges or reduces the light pattern that has passed through the refracting member 140 according to the magnification and projects the same onto the object.

In this manner, the light pattern projected onto the object by the projector 150 is captured by the image sensor 180. The image sensor 180 is arranged to capture the light pattern projected onto the object disposed in front of the projector 150. The light pattern is modified according to the surface shape of the object. The image sensor 180 captures a modified form of the light pattern irradiated onto the object so that the 3D shape of the object to be 3D scanned may be computed. A charge coupled device (CCD), complementary metal oxide semiconductor (CMOS), and the like may be used as the image sensor 180. The image captured by the image sensor 180 in this manner is transmitted to a separate computing device or to a storage device to be stored therein, and is used to compute the 3D shape. Pattern images may be captured using two or more image sensors 180 as needed. The 3D shape of the object may be computed by applying triangulation to the image captured by the image sensor 180.

Hereinafter, the operation of the single pattern shift projection optical system for a 3D scanner configured as described above will be described.

As described above, the controller 170 alternately turns off the two light sources 111 and 112. Light generated by each of the light sources 111 and 112 is projected in the form of a pattern while passing through the patterning member 130. The incident path member 120 transmits or reflects light generated by each of the two light sources 111 and 112, so that the light is incident on the patterning member 130 in the same direction.

Light incident on the patterning member 130 is projected onto the refracting member 140 in a striped pattern while passing through the slits provided in the patterning member 130.

As described above, the refracting member 140 of this embodiment is configured in the form of flat glass disposed to be tilted. Accordingly, with reference to FIG. 2, patterned light is refracted when entering and exiting the refracting member 140, respectively. At this time, the light entering the refracting member 140 and the light exiting the refracting member 140 have the same direction of travel, but refraction occurs upon entry and exit, respectively, thereby causing a lateral displacement of the path of the patterned light. When light travels through different media, the light refracts at the interface according to Snell's law, and the rate of refraction varies with wavelength. The phenomenon in which colors are separated due to changes in refractive index depending on the wavelength of light is referred to as chromatic dispersion.

Since the light generated by the light source 111 and the light generated by the 112 are red light and blue light having different wavelengths as described above, the respective colors of light experience different amounts of refraction while passing through the refracting member 140, thereby resulting in a difference d in lateral displacement. FIG. 2 shows the difference d in refraction and lateral displacement of red light and blue light. Since the wavelength of blue light is shorter than that of red light, the angle of refraction of blue light is greater than that of red light. As a result, since the lateral displacement of blue light is greater, the patterns caused by red light and blue light are shifted with respect to each other.

When such patterns from the light sources 111 and 112 are irradiated onto the target via the projector 150, the mutually shifted patterns are projected onto the target.

As a result, when the controller 170 alternately turns off the two light sources 111 and 112, the shifted patterns are alternately projected onto the object. As a result, the single pattern shift projection optical system for a 3D scanner of this embodiment generates two pattern images that are shifted with respect to each other. That is, the single pattern shift projection optical system for a 3D scanner of this embodiment may project two pattern-shifted or phase-shifted pattern images simply by alternately turning off the two light sources 111 and 112.

Accordingly, the light pattern irradiated by the projector 150 is projected onto the object to be scanned, and the image sensor 180 captures the pattern image projected onto the object. Using the image captured by the image sensor 180, the 3D shape of the object may be computed by triangulation.

At this time, projecting a plurality of pattern images onto the object as in this embodiment may produce accurate corresponding points. Compared to using a single pattern image, using a plurality of pattern images may produce a larger number of more accurate corresponding points, thereby resulting in higher resolution 3D data.

As described above, the present disclosure makes it possible to generate a pattern-shifted or phase-shifted pattern image with a very simple and unsophisticated structure using the principles of simple light source control and chromatic dispersion without using an optical modulator or a piezoelectric actuator. Accordingly, the structure of a 3D scanner may be simplified and miniaturized by using the single pattern shift projection optical system structure for a 3D scanner of the present disclosure. In addition, the fabrication cost of the 3D scanner of the present disclosure may be dramatically reduced. Since the single pattern shift projection optical system for a 3D scanner is designed with a relatively simple structure without using an optical modulator, power consumption is also reduced. As a result, using the single pattern shift projection optical system for a 3D scanner of the present disclosure makes it possible to easily develop a 3D scanner that may be used wirelessly, such as a dental scanner. In addition, since the single pattern shift projection optical system for a 3D scanner of the present disclosure may be designed with a relatively simple structure, restrictions in shape and structure may be reduced, thereby advantageously enabling the fabrication of 3D scanners having various shapes. For example, according to the present disclosure, a thin and long 3D scanner may be fabricated without much difficulty.

The present disclosure has been described above with reference to a preferred example, but the scope of the present disclosure is not limited to the form described and shown above.

For example, the previously described embodiment has been described as including the controller 170, the image sensor 180, the projector 150, and the like, but the single pattern shift projection optical system for a 3D scanner may be configured in a form without one or more of these elements. The present disclosure may be produced, sold, and used in a form in which only the plurality of light sources 111 and 112 described above, the patterning member 130, and the refracting member 140 are used to produce a plurality of mutually shifted patterns by chromatic dispersion. The present disclosure may be used by adding various optical devices to the single pattern shift projection optical system for a 3D scanner of this structure or by modifying the controller 170, the image sensor 180, the projector 150, or the like as needed.

In addition, the single pattern shift projection optical system for a 3D scanner including the two light sources 111 and 112 that emit blue light and red light, respectively, has been described above as an example, but the number of the light sources 111 and 112 and the wavelength of light generated by each of the light sources 111 and 112 may be varied as needed.

In addition, the structures of the light sources 111 and 112 may include light sources having various structures other than the single-wavelength LED lamp described above. For example, a laser diode (LD) lamp may be used as a light source in place of an LED lamp, and a light source that generates a single wavelength band of light, which is a combination of a lamp that generates a plurality of wavelengths of light rather than a single wavelength of light and a wavelength filter (or a dichroic filter), may also be used in the present disclosure.

In addition, the incident path member 120 may be configured as incident path members having various other structures, and in some cases, the single pattern shift projection optical system for a 3D scanner may be configured in a form that does not include the incident path member 120. For example, when the plurality of light sources 111 and 112 are disposed to emit light in the same direction, the separate incident path member 120 may not be required.

In addition, the refracting member 140 may have various shapes other than those described above. Various other forms of refracting members capable of refracting light at different angles of refraction depending on the wavelength of light generated by each light source, including a refracting member 240 of a single pattern shift projection optical system for a 3D scanner according to a second embodiment described below, may be used.

As described above, the present disclosure may be fabricated in a form without the projector 150, the controller 170, the image sensor 180, and the like, and the projector 150, the controller 170, the image sensor 180, and the like may be added to and used in the single pattern shift projection optical system for a 3D scanner according to the present disclosure according to the needs of the user who intends to use the present disclosure.

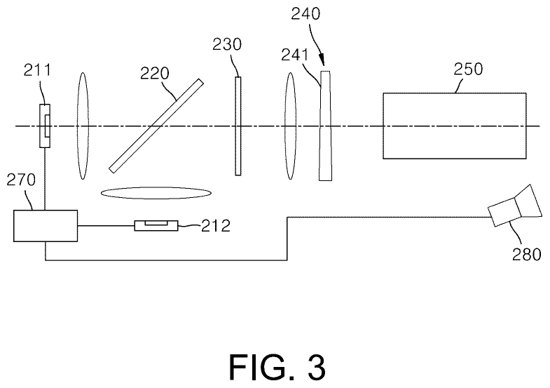

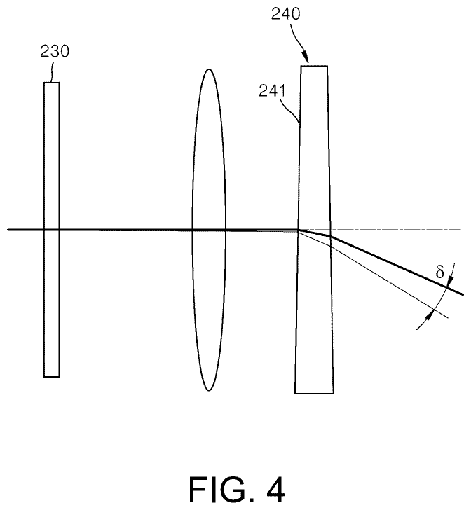

Next, with reference to FIGS. 3 and 4, the single pattern shift projection optical system for a 3D scanner according to the second embodiment of the present disclosure will be described.

FIG. 3 is a schematic diagram of the single pattern shift projection optical system for a 3D scanner according to the second embodiment of the present disclosure, and FIG. 4 is a diagram for explaining the operation of a portion of the single pattern shift projection optical system for a 3D scanner shown in FIG. 3.

The single pattern shift projection optical system for a 3D scanner according to the second embodiment includes light sources 211 and 212 and an incident path member 220 a patterning member 230, a controller 270, and an image sensor 280, the same as those of the single pattern shift projection optical system for a 3D scanner described with reference to FIGS. 1 and 2. The controller 270 and the image sensor 280 are elements that may be added or omitted as needed, as in the first embodiment. Since the major elements of the second embodiment described above are the same as those of the first embodiment, these elements are only shown with different numerals and detailed descriptions are omitted.

Unlike the refracting member 140 of the first embodiment, the refracting member 240 of the second embodiment is configured in the form of a thin prism. Accordingly, the refracting member 240 includes a tilted surface 241 that is made of a transparent material and disposed to be tilted with respect to the path of light passing through the patterning member 230.

Due to the structure of the refracting member 240 described above, an angular deviation δ occurs between an incident path and an exit path with respect to the refracting member 240 according to the wavelength of patterned light, as shown in FIG. 4. That is, unlike the first embodiment in which the lateral displacement occurred in the patterned light according to the wavelength, in the single pattern shift projection optical system for a 3D scanner of the second embodiment, the angular deviation δ according to the wavelength of the patterned light occurs due to the difference in the structure of the refracting member 240. That is, since blue light having a shorter wavelength is refracted at a larger angle than red light having a longer wavelength, the angle deviation δ is caused by the refracting member 240.

The projector 250 adjusts the path of each patterned light in which the angle deviation & occurs and projects the patterned light onto the object. The projector 250 enlarges or reduces the light pattern that has passed through the refracting member 240 according to the magnification and projects the same onto the object. The patterned light that has passed through the projector 250 is projected in the form of a pattern image onto the object to be 3D scanned.

As a result, when the controller 270 alternately turns off the light sources 211 and 212 that emit different wavelengths of light, two different pattern-shifted or phase-shifted pattern images may be produced due to chromatic dispersion by the refracting member 240.

As described above, the structure of the refracting member 240 may be modified in various manners as needed. For example, a combination of the refracting member 140 of the first embodiment and the refracting member 240 of the second embodiment may be used, and the refracting member capable of separating the optical path according to the wavelength of the patterned light may be used in various other structures.

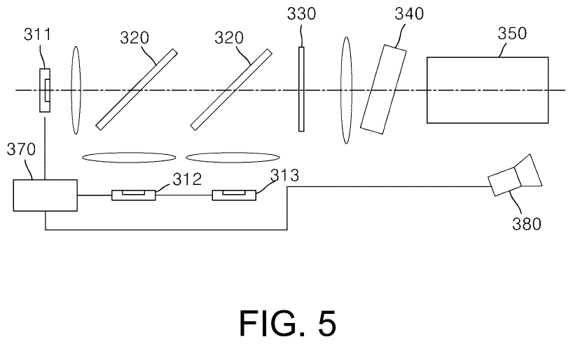

Next, with reference to FIG. 5, a single pattern shift projection optical system for a 3D scanner according to a third embodiment of the present disclosure will be described.

The single pattern shift projection optical system for a 3D scanner of the third embodiment differs from the first embodiment in that three light sources 311, 312, and 313 are provided, and the remaining elements are the same as those of the 3D scanner of the first embodiment. The controller 370 and the image sensor 380 are elements that may be added or omitted as needed, as in the first embodiment. Since the major elements of the third embodiment described above are the same as those of the first embodiment, these elements are only shown with different numerals and detailed descriptions are omitted.

The single pattern shift projection optical system for a 3D scanner of the third embodiment uses the light sources 311, 312, and 313 having three different wavelengths of red, green, and blue. The controller 370 sequentially turns off the three light sources 311, 312, and 313. The incident path member 320 is configured to adjust the optical path using various optical devices so that light generated by each of the three light sources 311, 312, and 313 is incident on a patterning member 330 in the same direction (i.e., a direction parallel to each other).

The refracting member 340 refracts patterned light at different angles of refraction depending on the wavelength of light generated by each light source 311, 312, and 313, and accordingly, three different pattern-shifted or phase-shifted pattern images are projected onto the object via the projector 350.

When the three pattern images are respectively projected onto the object irradiated by the projector 350 in this manner and then captured by the image sensor 380, the 3D shape of the object may be computed.

As described above, the number of the light sources 311, 312, and 313 may be varied as needed, so that any number of light sources having different wavelengths may be used. For example, a single pattern shift projection optical system for a 3D scanner of the present disclosure may be implemented using four light sources that emit red light, yellow light, green light, and blue light, respectively. In this case, four pattern-shifted or phase-shifted pattern images may be projected onto the object. As described above, the number of the light sources may be varied according to different wavelengths, and the controller may sequentially turn off these light sources, so that the same number of different pattern images as the number of the light sources may be projected onto the object.

The single pattern shift projection optical system for a 3D scanner according to the present disclosure, as in the first to third embodiments described above, may include at least one image sensor or be combined with the image sensor to form an oral scanner.

When an oral scanner is constructed according to the present disclosure, the dental scanner may be inserted into the oral cavity and scan teeth in a non-contact manner so as to be used to create a 3D model of the oral cavity including at least one tooth.

Accordingly, the oral scanner may be configured in a form that may be inserted into and removed from the oral cavity. Such an oral scanner scans the inside of a patient's oral cavity using at least one image sensor (e.g., an optical camera). The oral scanner may acquire surface information about the object as raw data to image the surface of the object, i.e., at least one of teeth in the oral cavity, the gum, and an artificial structure insertable into the oral cavity (e.g., an orthodontic appliance including a bracket and a wire, an implant, artificial teeth, and an orthodontic aid inserted into the oral cavity).

The image data acquired by the image sensor of the oral scanner configured by applying the present disclosure in this manner may be transmitted to an oral diagnostic device connected via a wired or wireless communication network and be used to compute a 3D shape. Such an oral diagnostic device may exist in the form of a server (or server device) for processing oral images.

The oral scanner described above may transmit raw data acquired by the oral scan to the oral diagnostic device. In this case, the oral diagnostic device may generate 3D shape data representing the oral cavity in three dimensions, based on the received raw data. The oral diagnostic device may analyze, process, display, and/or transmit the generated oral image.

In another example, the oral scanner may acquire raw data by the oral scan, directly process the acquired raw data to generate 3D shape data of the oral cavity, which is the object, and transmit the same to the oral diagnostic device. In this case, the oral diagnostic device may analyze, process, display, and/or transmit the received image.

In the oral scanner constructed according to the present disclosure, the oral scanner may include an L camera (not shown) corresponding to the left field of view and an R camera corresponding to the right field of view as image sensors in order to generate 3D shape data by an optical triangulation method. The oral scanner may acquire L image data corresponding to the left field of view and R image data corresponding to the right field of view from the L camera and the R camera, respectively. In this case, the L image data and R image data acquired by the L camera and the R camera, respectively, are image data in which the pattern images are projected onto the object by the single pattern shift projection optical system for a 3D scanner of the present disclosure described above.

When the oral scanner transmits raw data including the L image data and the R image data acquired in this manner to the communication interface of the oral diagnostic device, the communication interface 420 of the oral diagnostic device transmits the raw data to the processor. The processor generates 3D shape data representing the oral cavity in three dimensions, based on the received raw data. As described above, the single pattern shift projection optical system for a 3D scanner of the present disclosure may project a plurality of pattern-shifted or phase-shifted pattern images using only a relatively simple and unsophisticated structure using the principle of chromatic dispersion, so that the oral scanner according to the present disclosure may acquire high-resolution 3D shape data by producing a larger number of more accurate corresponding points.

The communication interface of the oral scanner and the oral diagnostic device may be implemented as various configurations capable of communicating with an external electronic device (e.g., an oral scanner, a server, or an external medical device) over a wired or wireless communication network. Such a communication interface may include at least one short-range communication module that communicates in accordance with communication standards such as Bluetooth, Wi-Fi, Bluetooth Low Energy (BLE), NFC/RFID, Wi-Fi direct, UWB, or Zigbee.

In addition, the communication interface may further include a long-range communication module that communicates with a server to support long-range communications in accordance with long-range communication standards. That is, the communication interface may include a long-range communication module that communicates over a network for Internet communication. In addition, the communication interface may include a long-range communication module that communicates over a communication network that complies with communication standards such as 3G, 4G, and/or 5G.

In addition, the communication interface may include at least one port for connection to an external electronic device (e.g., an oral scanner) through a cable to communicate with the external electronic device through the cable. For example, the communication interface may include a cable connection port, such as an HDMI port. In addition, the communication interface may communicate with a wired external electronic device through at least one port.

1. A single pattern shift projection optical system for a 3D scanner, comprising:
a plurality of light sources configured to emit light in different constant wavelength bands, respectively;
a patterning member configured to transmit a portion of the light generated by each of the plurality of light sources as patterned light; and
a refracting member disposed on a path of light passing through the patterning member and configured to refract and transmit the patterned light.

2. The single pattern shift projection optical system according to claim 1,
wherein the refracting member is a prism.

3. The single pattern shift projection optical system according to claim 1,
wherein the refracting member is a flat plate of transparent material disposed to be tilted with respect to the path of light passing through the patterning member.

4. The single pattern shift projection optical system according to claim 1,
wherein the refracting member is made of a transparent material and has a tilted surface disposed to be tilted with respect to the path of light passing through the patterning member.

5. The single pattern shift projection optical system according to claim 1,
further comprising an incident path member configured to adjust the path of light so that the light generated by each of the plurality of light sources is incident on the patterning member in a same direction.

6. The single pattern shift projection optical system according to claim 1,
further comprising a controller configured to control turning-on of the plurality of light sources.

7. The single pattern shift projection optical system according to claim 6,
wherein the controller sequentially turns off the plurality of light sources.

8. The single pattern shift projection optical system according to claim 6,
wherein the patterning member comprises a plurality of slits arranged parallel to each other at regular intervals.

9. The single pattern shift projection optical system according to claim 1,
wherein the plurality of light sources
comprise two light sources comprising a light source emitting red light and a light source emitting blue light.

10. The single pattern shift projection optical system according to claim 1,
wherein the plurality of light sources
comprise three light sources comprising a light source emitting red light, a light source emitting green light, and a light source emitting blue light.

11. The single pattern shift projection optical system according to claim 1,
wherein the plurality of light sources
comprise four light sources comprising a light source emitting red light, a light source emitting green light, a light source emitting blue light, and a light source emitting yellow light.

12. The single pattern shift projection optical system according to claim 1,
wherein each of the plurality of light sources
is an LED lamp.

13. The single pattern shift projection optical system according to claim 6,
further comprising an image sensor configured to capture an image of an object onto which the patterned light that has passed through the refracting member is projected.
